(19) United States
(12) Patent Application Publication
(10) Pub. No.: US 2021/0236245 A1
(43) Pub. Date: Aug. 5, 2021

(54) MEDICAL IMPLANT WITH DISCONTINUOUS OSSEOINTIGRATIVE SURFACE

(71) Applicant: University of Washington, Seattle, WA (US)

(72) Inventors: Alexander Pozhitzkov, Pasadena, CA (US); James D. Bryers, Seattle, WA (US); Eric Lindhahl, Seattle, WA (US); Daniel C. Chan, Seattle, WA (US)

(73) Assignee: University of Washington, Seattle, WA (US)

(21) Appl. No.: 17/235,433

(22) Filed: Apr. 20, 2021

Related U.S. Application Data
(62) Division of application No. 15/484,632, filed on Apr. 11, 2017.
(60) Provisional application No. 62/321,004, filed on Apr. 11, 2016.

(51) Int. Cl. A61C 8/00; A61C 8/02; A61C 13/00; A61F 2/00

(52) U.S. Cl. CPC A61C 8/0015; A61C 8/0006; A61F 2210/0009; A61C 13/0006; A61F 2/0077; A61C 8/0022

(57) ABSTRACT

A medical implant includes a base portion configured for implantation into a bone of a patient. The base portion is formed from an electrically insulating and biocompatible base material with retaining features on an outer surface of the base portion for gripping the bone in the patient. A plurality of titanium bands is positioned along the outer surface of the base portion.

### CROSS-REFERENCES TO RELATED APPLICATIONS

The present application claims the benefit under 35 USC § 119(e) of U.S. Provisional Appin. No. 62/321,004 filed Apr. 11, 2016; the full disclosure which is incorporated herein by reference in its entirety for all purposes.

### BACKGROUND

A dental implant is often implanted into a patient's jaw bone to replace a lost tooth, in order to restore function and aesthetics. While osseointegrated dental implants have been in commercial use since at least 1978, there are still problems related to their use. Issues such as peri-implantitis, possible titanium leaching, and corrosion of the titanium in implants are among the issues associated with titanium implants. Despite these issues, titanium implant surfaces remain heavily favored for their osteointegrative properties, strength, and flexibility.

Peri-implantitis is an inflammatory response that results in the destruction of soft tissue and bone around an implant. It impairs oral health-related quality of life in affected patients and is a major reason for the failure of implant-supported dental prostheses. Approximately one out of four patients with dental implants develops peri-implantitis within 11 years following implant placement. See, Daubert et. al. Prevalence and predictive factors for peri-implant disease and implant failure: a cross-sectional analysis, J Periodontol. 2015; 86: 337-47. Peri-implantitis has been associated with history of periodontitis, bacterial plaque, bleeding, bone level loss on the medium third of the implant, poor prosthetic fit, suboptimal screw joint, metal-ceramic restorations, with bacterial plaque, and with the presence of closely associated teeth or implants. More recently, high throughput DNA sequencing analysis has demonstrated that the microbial communities associated with peri-implantitis are distinct from peri-implant healthy communities and those found in periodontitis. Nevertheless, according to a recent systematic review, there is no consensus about the etiology of peri-implantitis and its relation to periodontitis. See Pesce et al., Peri-implantitis: a systematic review of recently published papers. Int J Prosthodont. 2014; 27: 15-25.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Various embodiments herein described relate to medical implants having multiple, separated regions of an osseointegrative, conductive material such as titanium on or exposed at an outer surface of a nonconductive, biocompatible material. Embodiments also relate to systems and methods for making such implants. In specific embodiments, the implants are dental implants having screw-form base portions designed for gripping bone upon implantation into a patient's gums. The base portion includes an outer surface with at least two titanium regions that are discontinuous and sufficiently separated so that a charge path does not form between the titanium regions.

Various embodiments of the medical implants herein described can be formed by providing a titanium coating on an outside surface of an nonconductive base material and then removing portions of the titanium layer by etching to form a discontinuous pattern. In some embodiments, a titanium layer can be applied to the nonconductive base material using a patterned mask to form a discontinuous pattern of titanium on the nonconductive base material. Still other embodiments can be formed by merging titanium particulates, or titanium bands, into a nonconductive base material precursor and then forming the mixed structure into the shape of the implant (e.g. a screw-form shape). Specific embodiments can include a mixture of titanium particulates embedded in a matrix, such as a ceramic matrix; or can include a series of titanium rings, bands, or washers embedded in a nonconductive matrix, and secured therein by heat treating (e.g. sintering) the resulting composite.

### BRIEF DESCRIPTION OF THE DRAWINGS

Various embodiments in accordance with the present disclosure will be described with reference to the drawings, in which:

FIG. 1 is a side view illustrating an example of a conventional medical implant having a base portion fully covered in a conductive surface and positioned in a patient's gums;

FIG. 2 is a side view illustrating a first example of a medical implant, which has a base portion with a series of discontinuous titanium surface regions, in accordance with embodiments of the present invention;

FIG. 3 is a side view illustrating a second example of a medical implant, which has a base portion with a series of distinct titanium regions positioned throughout the base portion, in accordance with embodiments;

FIG. 4 is a side view illustrating a third example of a medical implant, which has a base portion with a series of distinct titanium bands positioned along a length of the implant, and accordance with embodiments;

FIG. 5 is a simplified block diagram illustrating a system, in accordance with embodiments, which can be used to design and fabricate a medical implant in accordance with any of FIGS. 2-4;

FIG. 6 illustrates an example process, in accordance with embodiments, which can be used to fabricate a medical implant in accordance with FIGS. 2-4;

FIG. 7 illustrates an example process, in accordance with embodiments, which can be used to fabricate a medical implant base portion in accordance with FIG. 2;

FIG. 8 illustrates another example process, in accordance with embodiments, which can be used to fabricate a medical implant base portion in accordance with FIG. 2;

FIG. 9 illustrates another example process, in accordance with embodiments, which can be used to fabricate a medical implant base portion in accordance with FIG. 3; and

FIG. 10 illustrates another example process, in accordance with embodiments, which can be used to fabricate a medical implant base portion in accordance with FIG. 4.

### DETAILED DESCRIPTION

Titanium is currently considered the gold standard for dental implants due to promoting excellent osseointegration rates, in addition to the strength and flexibility of the material. While many other materials have been explored as substitutes, no suitable substance has been found that competes directly in terms of osseointregration, strength, and flexibility. Thus, titanium continues to be the most widely used material in dental implantology. Although titanium is prone to gradual corrosion when implanted in a patient, this corrosion typically slows or halts entirely as a titanium implant builds up an oxidation layer following implantation. Titanium is, however, prone to more rapid corrosion in the context of dental implants with associated peri-implantitis.

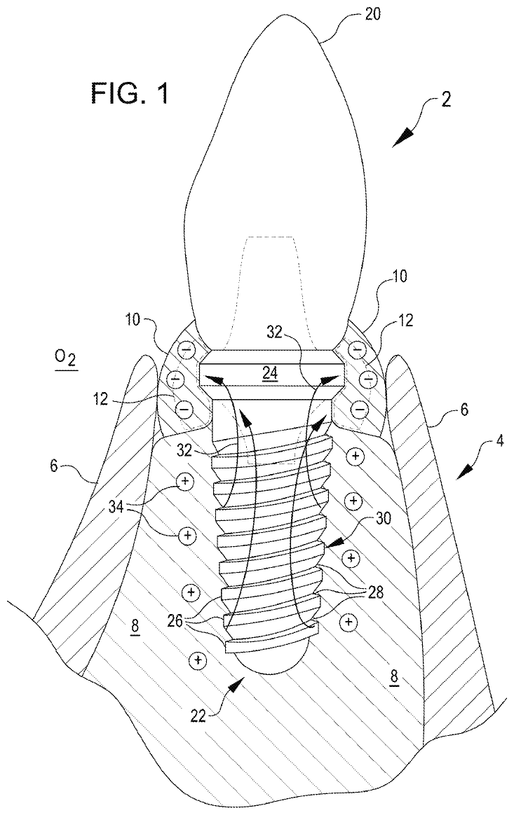

Turning now to the drawing figures in which like reference numbers refer to like elements, FIG. 1 is a side view illustrating an example of a conventional medical implant 2 having a base portion 22 fully covered in a conductive outer surface 30 and positioned in a patient's gums 4. The implant has a crown portion 20, base portion 22, and an abutment 24 connecting the crown portion 20 and the base portion 22. The base portion 22 has a screw thread 26, which has a minor diameter channel 28, which is used to grip a jaw bone 8 of the patient. The base portion 22 is embedded in the jaw bone 8, which is surrounded by gum tissue 6. A boundary region 10 containing a combination of tissue, plaque burden, and a pathogenic microbial biofilm, is typically formed near the upper boundary of the jaw bone 8. Following implantation of the base portion 22 into the jaw bone 8, an aerobic zone 12 typically forms in the boundary region 10, in which oxygen is metabolized by microorganisms in a process that results in a negative charge throughout the boundary region 10. The conductive outer surface 30, which is typically titanium, enables charge paths 32 to flow along the outer surface, creating local pockets of positive charge 34 adjacent the implant base 22. This transmission of electrical charge accelerates the ordinarily, gradual oxidation of the titanium throughout the outer surface 30, and leads to gradual corrosion of the implant base 22. Furthermore, the presence of positive charge proximate to the jaw bone 8 is associated with bone resorption, indicating a possibility that the positive charge inhibits healthy bone formation or maintenance surrounding the implant base 22.

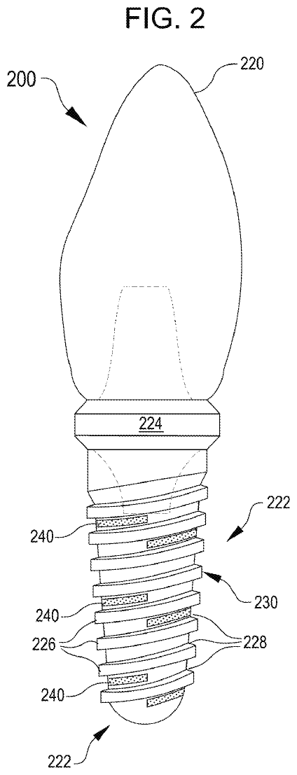

Embodiments described herein relate to improved medical implants that provide some of the benefits of a titanium outer surface such as beneficial osseointegration rates while mitigating the oxidation effect observed with reference to conventional titanium implants. As described below with reference to FIGS. 2-4, a medical implant can include a base portion with retaining features for connecting the implant with bone, along with multiple discontinuous titanium regions at an outer surface of the implant. Each respective titanium region confers benefits of improved biocompatibility and integration into the bone, while separation and electrical isolation of each respective titanium region from each other titanium region inhibits the development of long charge paths between one portion of the implant base and another, e.g. between a region near an aerobic zone producing negative charge and a region embedded in an anaerobic zone.

As described herein, discontinuous titanium regions can be embedded in or formed on an outer surface of the base portion of a medical implant, and in specific embodiments described herein, on a dental implant. Suitable base portions can be composed of a nonconductive, i.e. electrically insulating and structurally rigid base material. Suitable base materials can include various high-strength ceramics, but can include, in alternative embodiments, composite materials, structural polymers, or the like. Suitable base materials can include, but are not limited to: single-crystal alumina ceramic; porcelain; polycrystalline alumina ceramic; bioactive glass or glass coatings including SiO₂, Ca, Na₂OH, H, and P coatings; zirconium dioxide; polymethyl-methacrylate (PMMA); vitreous carbon, and the like.

FIG. 2 is a side view illustrating a first example of a medical implant 200 having a base portion 222 with a series of discontinuous titanium surface regions 240 positioned on the outer surface 230 of the base portion, in accordance with embodiments. The implant 200 includes a crown portion 220 suitable for use as an artificial tooth, an abutment 224, and a base portion 222 connected with the crown portion 220 by the abutment 224. While the illustrated base portion 222 has a screw thread 226 with a minor diameter channel 228, the principles described herein are applicable to implants using alternative retention features, such as rings, hooks, barbs, textured surfaces, or the like.

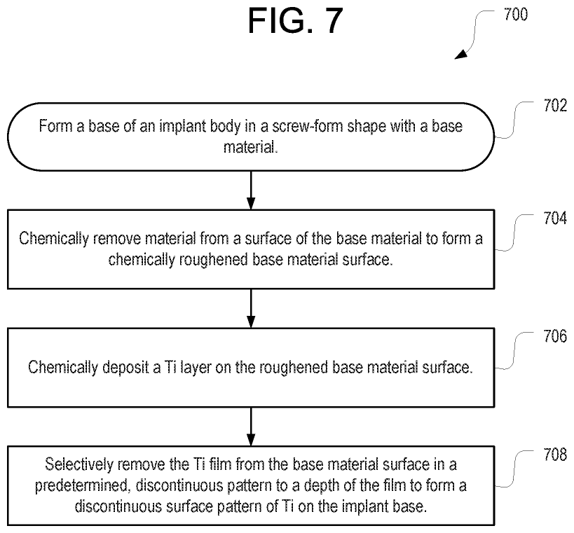

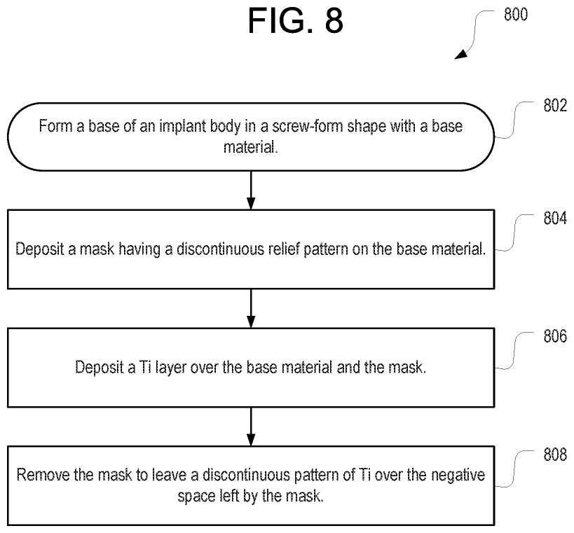

Multiple discontinuous regions 240 including a titanium coating are positioned on the outer surface 230, and are separated by regions of a nonconductive material making up the base portion 222 without the titanium coating. According to some embodiments, the discontinuous titanium regions 240 may be disposed within the minor diameter channel 228 of the screw thread 226. In alternative embodiments, the discontinuous titanium regions 240 may be positioned on portions of the major diameter of the screw thread 226, or may be positioned both on portions of the major diameter of the screw thread 226 and portions of the minor diameter channel 228, provided the titanium regions 240 are sufficiently separated to prevent charge from passing readily between the discontinuous titanium regions 240. In specific embodiments, the titanium regions 240 are separated by at least 0.1 mm. The number of titanium regions 240 may vary depending on region size, implant depth, and on the specific separation between regions. According to embodiments, at least two titanium regions 240 are present on the outer surface 230. Preferably, each discontinuous titanium region 240 has a vertical extent ranging from about 1 mm to about 3 mm. According to some embodiments, the outer surface 230 is substantially covered in titanium bands that are interrupted by discrete openings at intervals ranging from about 0.1 mm to 1 mm, such that the bands are electrically isolated from each other. The bands 240 can be formed of thin titanium layers embedded in or deposited on the outer surface 230 of the implant base 222. Suitable methods for embedding or depositing the bands 240 are described below with reference to FIGS. 7 and 8. An overall surface area of the outer surface 230 of the base portion 222 can be covered with the discontinuous regions 240 from about 10% of the surface area to about 90% of the surface area.

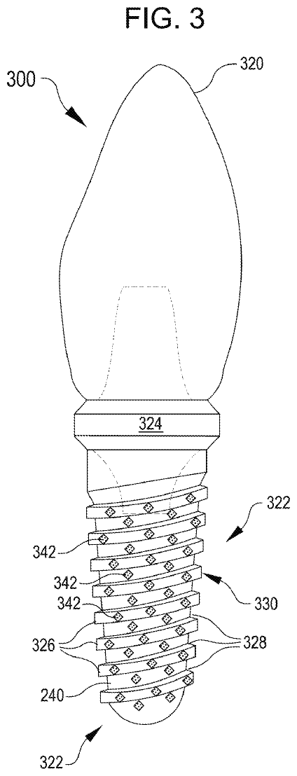

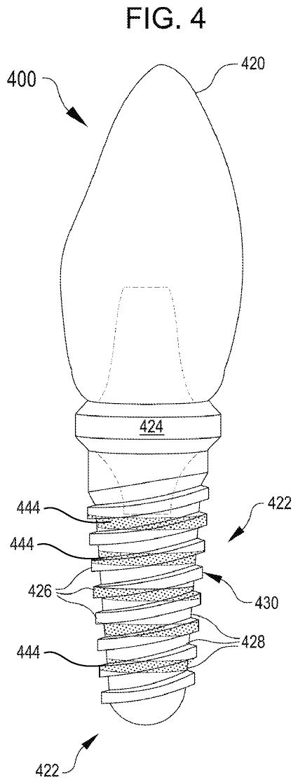

According to various embodiments, discontinuous regions of titanium can be added to an outer surface of a base portion of an implant according to methods other than bonding the titanium regions to the outer surface. For example, according to some embodiments, titanium particles or titanium structures can be integrated into the base portion and exposed at the outer surface, as described below with reference to FIGS. 3 and 4. In accordance with embodiments described above, embodiments having embedded titanium particles or titanium structures can include any suitable number of distinct titanium regions on the outer surface, provided the regions are generally electrically isolated in a vertical direction along the implant, i.e. separated by at least 0.1 mm.

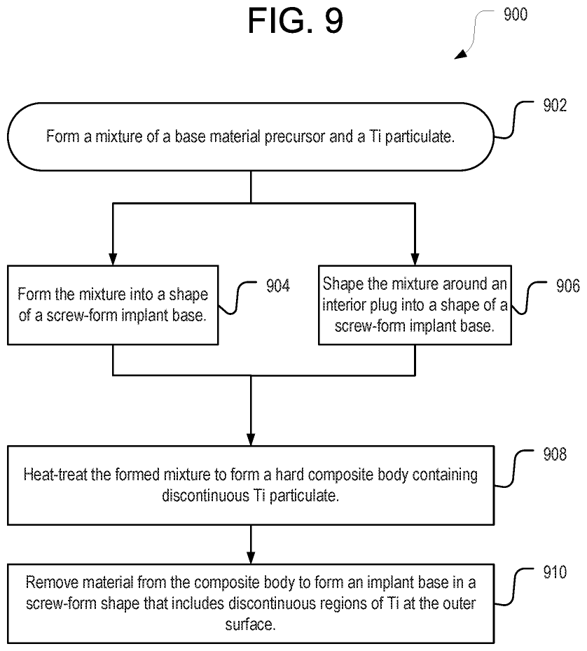

FIG. 3 is a side view illustrating a medical implant 300 having a crown portion 320, abutment 324, and base portion 322. The base portion 322 includes a series of small, discontinuous regions 342 formed of titanium positioned throughout an outer surface 330 of the base portion, in accordance with embodiments. The discontinuous regions 342 can be formed by embedding titanium particles throughout the base portion 322, and causing the titanium particles to be exposed at the outer surface 330. The discontinuous regions 342 can be speckled throughout the screw thread 326, which includes a minor diameter channel 328 and serves as a retention feature of the base portion 322, or throughout any other suitable retention features, such that the discontinuous regions come into contact with the jaw bone when the base portion 322 is implanted in the jaw bone. One suitable method of fabricating the medical implant 300 is described below with reference to FIG. 9. Although some portion of the titanium particles forming the discontinuous regions 342 may be in contact with one or more neighboring titanium particles, the titanium regions 342 can be formed so that sufficient numbers of titanium particles or groups of adjacent titanium particles are sufficiently electrically isolated from adjacent titanium particles or groups of titanium particles to act as multiple, disconnected regions of titanium disposed on the outer surface 330. An overall surface area of the outer surface 330 of the base portion 322 can be covered with the discontinuous regions 342 from about 10% of the surface area to about 90% of the surface area.

FIG. 4 is a side view illustrating a medical implant 400 having a crown portion 420, abutment 424, and base portion 422 connected with the crown portion via the abutment, with a series of discontinuous titanium bands 444 positioned along a length of the base portion of the implant, in accordance with embodiments. The titanium bands 444 can be formed by embedding titanium structures, e.g. washers or rings, within at least the outer surface 430 of the implant 400 prior to fabricating screw thread 426, which includes a minor diameter channel 428, or other retention features of the implant, on the outer surface. Similar to embodiments described above with reference to FIGS. 2-3, an overall surface area of the outer surface 430 of the base portion 422 can be covered with the discontinuous regions 444 from about 10% of the surface area to about 90% of the surface area. In specific embodiments, the number of discontinuous regions 444 can vary from as few as two regions, to an upper limit defined by a minimum separation distance of about 0.1 mm, a minimum vertical dimension of each region of about 1 mm, and the overall length of the base portion 422.

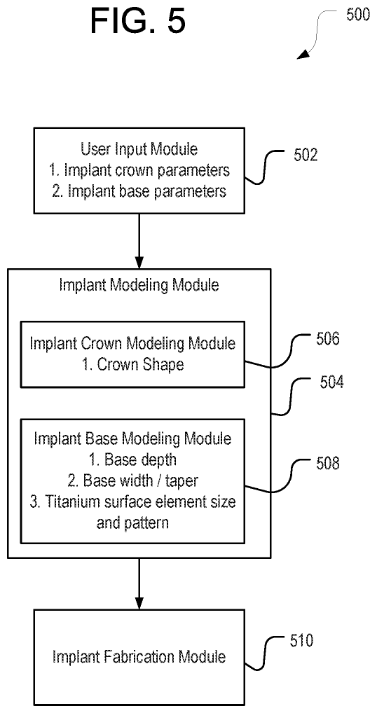

FIG. 5 is a simplified block diagram illustrating a system 500 for designing and fabricating a medical implant in accordance with any of FIGS. 2-4, in accordance with embodiments. The system 500 can include various components of physical hardware, including one or more processors and non-transitory memory for storing instructions thereon. The system 500 includes a user input module 502 for obtaining, receiving, and/or processing biometric data of a patient and for generating data indicative of patient needs. For example, the user input module can receive data concerning available space in a patient's jaw between existing teeth, and concerning an available or medically optimal depth for anchoring the implant. An implant modeling module 504 can receive data from the user input module 502, and process the data in order to define parameters of the medical implant. For example, an implant crown modeling module 506 can operate to define the shape of the crown of the implant. In the case of a dental implant, the implant crown modeling module 506 can determine at least a shape, a height, and a width of the implant crown to correspond with the patent's needs. Likewise, an implant base modeling module 508 can receive data from the user input module 502 and process the data to determine geometry of the base portion of the implant. This modeling can include determining an available depth, and determining a length, width, taper, thread count, threat depth, and other comparable attributes of the base portion of the implant. The implant modeling module 504 can pass data to an implant fabrication module 510 for producing the implant. In some cases, the implant fabrication module 510 can produce some or all of the implant directly, e.g. via 3D printing or the like. In some cases, the implant fabrication module can include multiple fabrication components including, e.g., surface deposition and/or chemical etching apparatuses, sintering apparatuses, machining apparatuses including automated or computer-numerically-controlled machining, and the like.

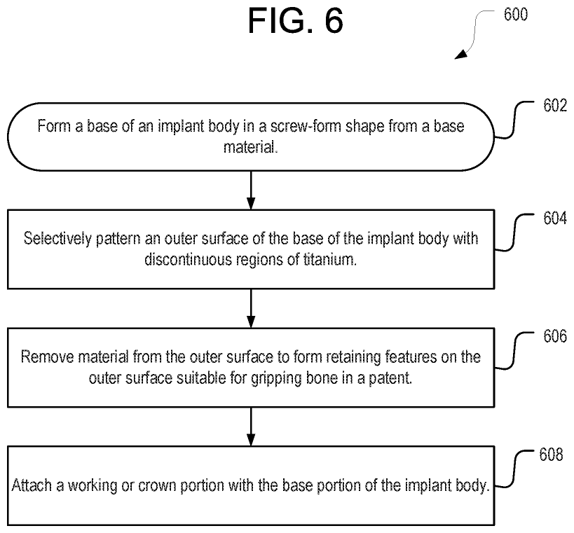

FIGS. 6-10 describe example processes (600, 700, 800, 900, 1000) for fabricating a medical implant such as the implants of FIGS. 1-4. Aspects of the processes 600, 700, 800, 900, 1000 can be performed, in some embodiments, in conjunction with a system such as system 500 described with reference to FIG. 5.

FIG. 6 illustrates an example process 600, in accordance with embodiments, for making a medical implant in accordance with any of FIGS. 2-4. The process 600 includes forming a base portion of an implant body in a screw-form shape from a suitable base material (act 602). An outer surface of the base portion can be selectively patterned with titanium to form multiple discontinuous regions of titanium thereon (act 604) so that the regions are electrically isolated from each other. Suitable methods of patterning the outer surface with titanium are described below with reference to FIGS. 7-10. Processes disclosed below with reference to FIGS. 7-10 may be combined with process 600, or with one another, except where explicitly contraindicated. Following the patterning of the outer surface with titanium, material can be removed from the outer surface to form retaining features for gripping bone and retaining the implant in a patient (act 606). Suitable retaining features on the outer surface can include various structures including but not limited to: threads, channels, barbs, and the like. In embodiments where the implant is formed of separate, connectible crown and base portions, the working or crown portion of the implant can then be connected with the base portion to form an entire implant body (act 600). In accordance with embodiments, connecting the crown and base portions can include forming a connection via an abutment.

FIG. 7 illustrates a process 700, in accordance with embodiments, for fabricating a medical implant in accordance with FIG. 2. The process 700 includes forming an implant body with predetermined overall shape parameters into a screw-form shape with a nonconductive base material (act 702). An outer surface of the base material can be roughened, chemically treated, (e.g. acid-washed via HCl or comparable solvent,) or otherwise treated to encourage bonding of titanium to the surface (act 704). A fabrication module, such as a vapor deposition or comparable apparatus, can be used to coat the base material with a layer of titanium on the outer surface (act 706). Various processes suitable for depositing a titanium layer can include physical vapor deposition, electro-catalyzed deposition from an ionic fluid, and deposition by thermal evaporation of a titanium-containing fluid, or comparable process. Once applied, the thin titanium layer can be selectively removed down to the base material of the implant body in a pattern that leaves multiple, electrically isolated titanium regions on the outer surface of the implant body (act 708). Suitable techniques for removing portions of the titanium layer can include chemical etching, e.g. using a photoresistive mask or selectively placed polymer mask in combination with HF acid washing or similar; or various forms of mechanical removal, such as but not limited to machining or ablation.

FIG. 8 illustrates another process 800, in accordance with embodiments, for making a medical implant in accordance with FIG. 2. In an embodiment, the process 800 includes forming an implant body with predetermined overall shape parameters into a screw-form shape with a nonconductive base material (act 802). Optionally, an outer surface of the base material can be roughened or otherwise treated to encourage bonding of titanium to the surface. A mask can then be deposited on the outer surface in a pattern having negative space that defines a discontinuous pattern on the surface (804). A titanium layer can be deposited on the combined outer surface and mask, so that the titanium layer bonds to the outer surface within the portions of the mask defining the discontinuous pattern (act 806) using any suitable titanium deposition method as discussed above. The mask can subsequently be removed along with excess titanium material to leave the discontinuous pattern of titanium bonded to the outer surface (act 808). Suitable methods of masking the outer surface can include using polymer films as masking layers, for example, or any other suitable masking layer. Methods for removing the masking layer can include chemical means such as solvent treatment, thermal means, or the like.

FIG. 9 illustrates a process 900, in accordance with embodiments, for fabricating a medical implant in accordance with FIG. 3. In an embodiment, the process 900 includes forming a mixture of a base material precursor and titanium particles (act 902). Suitable base material precursors include various malleable and/or uncured precursors for the nonconductive base portion of the implant body, including but not limited to ceramic precursor, polymer matrix precursor, and the like. The mixture, including titanium particles suspended therein, can then be formed into the shape of the base of the implant body, e.g. into a screw-form shape suitable for the base of the implant body (act 904). Optionally, the mixture can instead be shaped around an interior plug that partly defines the shape of the implant base (act 906), thus constraining titanium particles to a layer near the surface of the implant body. The formed mixture can then be treated to form a hard composite body, i.e. by heat treatment such as but not limited to sintering, to form an implant base with suspended titanium particles at or near the outer surface thereof (act 908). The hard composite body may be further processed by removing an outer layer from the outer surface in order to expose some portion of the titanium particles and/or to remove any residue of the heat treatment step (act 910).

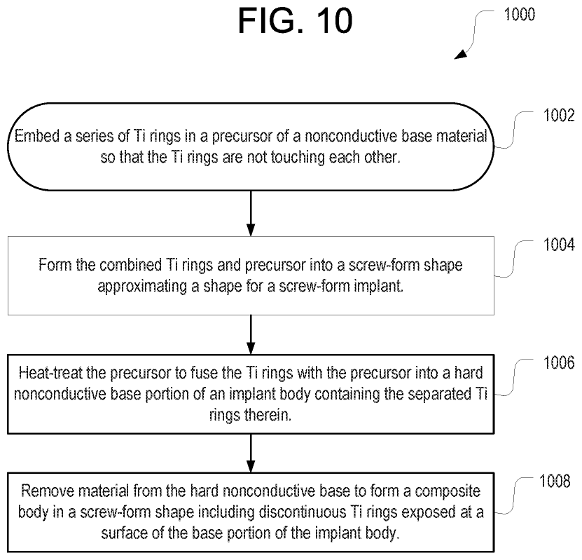

FIG. 10 illustrates a process 1000, in accordance with embodiments, for fabricating a medical implant in accordance with FIG. 4. The process 1000 includes forming a nonconductive base material precursor, such as a ceramic precursor, with a series of titanium rings in series with the titanium rings not in contact with one another (act 1002). The combined precursor and titanium rings can be formed into a screw-form shape approximating a shape for a screw-form implant (act 1004). The shaped combination of the precursor and titanium rings can be heat-treated to form a hard composite body defining a nonconductive base portion of an implant body with the series of separated titanium rings suspended therein, the rings extending near to or at an outer surface thereof (act 1006). Optionally, material can be removed from the hard composite body of the nonconductive base portion in order to refine the screw-form shape of the base portion, to expose portions of the titanium rings, to remove residue, or any combination of the above (act 1008).

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. -20. (canceled)

21. A dental implant, comprising:
a crown portion; and
a base portion connected with the crown portion and at least partially formed of an electrically insulating and biocompatible base material, the base portion having at least one screw thread formed in an outer surface of the base portion;
wherein a plurality of titanium bands is positioned along the outer surface of the base portion, wherein each titanium band of the plurality of titanium bands is spaced apart and electrically insulated from each other titanium band of the plurality of titanium bands along a length direction of the base portion by the electrically insulating and biocompatible base material, and wherein each titanium band of the plurality of titanium bands is radially continuous around the outer surface of the base portion and extends across the at least one screw thread.

22. The dental implant of claim 21, wherein each titanium band of the plurality of titanium bands is radially continuous around an entire circumference of the outer surface of the base portion.

23. The dental implant of claim 21, wherein each titanium band of the plurality of titanium bands extends across a minor diameter channel defined in the outer surface by the at least one screw thread.

24. The dental implant of claim 21, wherein each titanium band of the plurality of titanium bands is separated from each other titanium band of the plurality of titanium bands along the length direction by 0.1 mm to 1.0 mm.

25. The dental implant of claim 21, wherein each titanium band of the plurality of titanium bands has a vertical dimension along the length direction of 1.0 mm to 3.0 mm.

26. The dental implant of claim 21, wherein each titanium band of the plurality of titanium bands comprises a titanium washer or a titanium ring embedded in the outer surface of the base portion.

27. The dental implant of claim 21, wherein each titanium band of the plurality of titanium bands comprises a titanium surface coating bonded to the electrically insulating and biocompatible base material.

28. The dental implant of claim 21, wherein each titanium band of the plurality of titanium bands comprises titanium particles embedded in the electrically insulating and biocompatible base material.

29. The dental implant of claim 21, wherein each titanium band of the plurality of titanium bands comprises a layer of titanium surface coating bonded to the electrically insulating and biocompatible base material.

30. The dental implant of claim 21, wherein an abutment connects the base portion with the crown portion.

31. The dental implant of claim 21, wherein the plurality of titanium bands covers 10% to 90% of the outer surface of the base portion.

32. The dental implant of claim 21, wherein the electrically insulating and biocompatible base material comprises a ceramic.

33. The dental implant of claim 21, wherein the electrically insulating and biocompatible base material comprises one or more of a single-crystal alumina ceramic; a porcelain; a polycrystalline alumina ceramic; a bioactive glass; a zirconium dioxide; a polymethyl-methacrylate; or a vitreous carbon.

34. A dental implant, comprising:
a crown portion; and
a base portion connected with the crown portion and at least partially formed of an electrically insulating and biocompatible base material, the base portion having at least one screw thread formed in an outer surface of the base portion;
wherein a plurality of titanium bands is positioned along the outer surface of the base portion, wherein each titanium band of the plurality of titanium bands is spaced apart and electrically insulated from each other titanium band of the plurality of titanium bands along a length direction of the base portion by the electrically insulating and biocompatible base material, and wherein each titanium band of the plurality of titanium bands is radially continuous around an entire circumference of the outer surface of the base portion,
wherein each titanium band of the plurality of titanium bands extends across the at least one screw thread and across a minor diameter channel defined in the outer surface by the at least one screw thread.

35. The dental implant of claim 34, wherein each titanium band of the plurality of titanium bands is separated from each other titanium band of the plurality of titanium bands along the length direction by 0.1 mm to 1.0 mm.

36. The dental implant of claim 35, wherein each titanium band of the plurality of titanium bands has a vertical dimension along the length direction of 1.0 mm to 3.0 mm.

37. The dental implant of claim 36, wherein the plurality of titanium bands covers 10% to 90% of the outer surface of the base portion.

38. The dental implant of claim 37, wherein each titanium band of the plurality of titanium bands comprises a titanium washer or a titanium ring embedded in the outer surface of the base portion.

39. The dental implant of claim 37, wherein each titanium band of the plurality of titanium bands comprises a titanium surface coating bonded to the electrically insulating and biocompatible base material.

40. The dental implant of claim 37, wherein each titanium band of the plurality of titanium bands comprises titanium particles embedded in the electrically insulating and biocompatible base material.
